(19) United States
(12) Patent Application Publication
(10) Pub. No.: US 2024/0149063 A1
(43) Pub. Date: May 9, 2024

(54) TRANSCUTANEOUS AURICULAR VAGAL NERVE STIMULATION FOR SUBARACHNOID HEMORRHAGES

(71) Applicant: Washington University, St. Louis, MO (US)

(72) Inventors: Eric Leuthardt, St. Louis, MO (US); Anna Huguenard, St. Louis, MO (US)

(21) Appl. No.: 18/548,755

(22) PCT Filed: Mar. 4, 2022
(86) PCT No.: PCT/US2022/018864

§ 371 Date: Sep. 1, 2023

Related U.S. Application Data
(60) Provisional application No. 63/156,792, filed on Mar. 4, 2021.

(51) Int. Cl. A61N 1/36; A61N 1/04; A61N 1/05

(52) U.S. Cl. CPC A61N 1/36053; A61N 1/0456; A61N 1/0556; A61N 1/36031; A61N 1/36034; A61N 1/36036; A61N 1/36135; A61N 1/36157

(57) ABSTRACT

A method of treating an inflammation in a patient is provided. The method includes stimulating, in a patient suffering from inflammation, the patient's vagus nerve with an electrical signal to achieve a therapeutic effect for treating the inflammation. The signal current is 0.4 mA, the pulse width is 250 μs, the signal frequency is 20 Hz, and the signal on-time is twenty minutes. The method also includes re-stimulating the patient's vagus nerve a second time within 24 hours of the first stimulation. The electrical signal for a first stimulation and the second stimulation are the same.

### CROSS REFERENCE TO RELATED APPLICATIONS

This application claims the benefit of and priority to U.S. Provisional Patent Application No. 63/156,792, filed Mar. 4, 2021, which is hereby incorporated by reference in its entirety.

### BACKGROUND

The present disclosure generally relates to compositions and methods of treatment of spontaneous subarachnoid hemorrhages.

Between 3-5% of adults harbor an intracranial aneurysm, and 18-30% of those individuals have more than one aneurysm. Despite more frequent detection of unruptured aneurysms in the general population, many patients still present initially with a subarachnoid hemorrhage (SAH). For patients presenting with SAH, the mortality rate is 10-25%, with an additional 30% of patients suffering permanent disability. Following SAH, there is a significant risk for early brain injury and edema, cerebral vasospasm, and delayed cortical ischemia that all contribute to the high morbidity to these patients.

The pathophysiology of aneurysm formation and rupture is complex and influenced by genetic and environmental factors. There is evidence that systemic and local inflammation may promote aneurysm formation and rupture, and leads to poorer outcomes following SAH. T-cell and macrophage-mediated inflammation can mediate some histological changes within the vascular wall that leads to aneurysm formation, and macrophage infiltrates in the walls of ruptured aneurysms likely contribute to their fragility. Elevated levels of inflammatory mediators, complement, and vascular cell adhesion molecule-1 (VCAM-1) have also been demonstrated in aneurysms, compared to non-aneurysmal intracranial vessels. In aneurysms, cathepsin G, a serine protease produced primarily in neutrophils, can be found at the site of rupture, implicating neutrophils in the acute rupture process.

Following SAH, blood within the subarachnoid space triggers a local and systemic inflammatory response. Studies show that after SAH, there are increases in IL-1b, IL-6, IL-1, and TNF-α, within the CSF, increases in IL-1, IL-23, IL-17, and ICAM-1 in the serum, and increases in p-38 and p-MAPK in brain tissue. There is also evidence that inflammatory markers are correlated with patient outcomes. Elevated IL-6 has been associated with increased risk for vasospasm and poorer outcomes. Elevated IL-1b, IL-18, and TNF-α in the CSF are associated with cerebral edema and acute hydrocephalus. There is also evidence that the degree of leukocytosis alone on admission following SAH is associated with worse modified Rankin scale scores (mRS) on discharge.

A new avenue of research has aimed to better understand, and eventually target, inflammatory pathways to improve outcomes after SAH, with numerous anti-inflammatory interventions trialed in humans. In smaller enrollment studies, there has been some promise of outcome improvement with Cyclosporin A and various types of steroids (methylprednisolone, hydrocortisone, and dexamethasone). Other medications demonstrated no impact on overall outcomes, like Clazosentan, Cilostazol, and IL-1 antagonists. In larger trials with >1000 patients, Simvastatin, Aspirin, non-steroidal anti-inflammatory medications, and thienopyridines all demonstrated no improvement in outcomes. Therefore, a combination of medication side effects and lack of efficacy have prevented many anti-inflammatory medications from broader use in SAH.

It would be desirable to have an improved method for reducing inflammation to improve outcomes after SAH, while avoiding many medicinal side effects.

### BRIEF DESCRIPTION

In a first aspect, a method of treating an inflammation in a patient is provided. The method includes stimulating, in a patient suffering from inflammation, the patient's vagus nerve with an electrical signal to achieve a therapeutic effect for treating the inflammation. The signal current is 0.4 mA, the pulse width is 250 μs, the signal frequency is 20 Hz, and the signal on-time is twenty minutes. The method also includes re-stimulating the patient's vagus nerve a second time within 24 hours of the first stimulation. The electrical signal for a first stimulation and the second stimulation are the same.

In a second aspect, a system for treating an inflammation in a patient is provided. The system includes an electrical stimulation device including one or more electrodes. The electrical stimulation device is configured to provide an electrical current to a patient's vagus nerve with an electrical signal to achieve a therapeutic effect for treating the inflammation. The signal current is 0.4 mA, the pulse width is 250 μs, the signal frequency is 20 Hz, and the signal on-time is twenty minutes.

In a third aspect, an electrical stimulation device is provided. The electrical stimulation device includes one or more electrodes. The electrical stimulation device is configured to provide an electrical current to a patient's vagus nerve with an electrical signal to achieve a therapeutic effect for treating inflammation. The signal current is 0.4 mA, the pulse width is 250 μs, the signal frequency is 20 Hz, and the signal on-time is twenty minutes.

### BRIEF DESCRIPTION OF THE DRAWINGS

Those of skill in the art will understand that the drawings, described below, are for illustrative purposes only. The drawings are not intended to limit the scope of the present teachings in any way.

FIG. 1 illustrates a system for providing vagal nerve stimulation to a patient in accordance with at least one embodiment.

FIG. 2 illustrates placement of electrodes for non-invasive transcutaneous vagus nerve stimulation using the system shown in FIG. 1.

FIG. 3 illustrates a process for providing vagal nerve stimulation using the system shown in FIG. 1.

FIG. 4 illustrates an example configuration of a client system shown in FIG. 3, in accordance with one embodiment of the present disclosure.

There are shown in the drawings arrangements that are presently discussed, it being understood, however, that the present embodiments are not limited to the precise arrangements and are instrumentalities shown. While multiple embodiments are disclosed, still other embodiments of the present disclosure will become apparent to those skilled in the art from the following detailed description, which shows and describes illustrative aspects of the disclosure. As will be realized, the invention is capable of modifications in various aspects, all without departing from the spirit and scope of the present disclosure. Accordingly, the drawings and detailed description are to be regarded as illustrative in nature and not restrictive.

### DETAILED DESCRIPTION

In various aspects, devices and methods of treatment for subarachnoid hemorrhages (SAHs) are disclosed. In some aspects, the method includes administering vagal nerve stimulation (VNS) to a patient in need. In various aspects, VNS may be administered using any suitable method including, but not limited to, cervical neck dissection and placement of a cuff electrode directly on the nerve, and non-invasive transcutaneous stimulation of the auricular branch of the vagus nerve. In one aspect, the transcutaneous stimulation of the auricular branch of the vagus nerve is implemented using a portable TENS (transcutaneous electrical nerve stimulation) unit connected to two ear clip electrodes positioned in an ear of the subject. Without being limited to any particular theory, the external ear is an effective position for non-invasive stimulation of the vagus nerve, where the auricular branch travels in the pinna of the ear. In one aspect, the ear clips used for the VNS treatment are positioned along the concha of the ear.

### Therapeutic Methods

Aneurysmal spontaneous subarachnoid hemorrhage (SAH) is a disease with both high mortality and morbidity. Inflammation plays an important role in morbidity following an SAH. Despite extensive research, few interventions have consistently demonstrated improved outcomes in these patients. Transcutaneous auricular vagus nerve stimulation (taVNS) provides a novel, non-pharmacologic, non-invasive approach to immunomodulations with the potential to improve outcomes in SAH. Vagal nerve stimulation (VNS) allows for more global regulation of the parasympathetic system rather than targeting a single inflammatory pathway like prior pharmaceutical approaches. Provided is a method of treating, preventing, or reversing an inflammatory response following a spontaneous subarachnoid hemorrhage (SAH) to improve clinical outcomes in those patients. The method includes administration of a therapeutically effective amount of vagal nerve stimulation (VNS) to treat, prevent, reduce, or reverse an inflammatory response following a spontaneous subarachnoid hemorrhage (SAH).

Vagus nerve stimulation has been shown to reduce inflammation. Substantial work has demonstrated that products of infection or injury activate sensory neurons traveling to the brainstem in the vagus nerve. The arrival of these incoming signals generates action potentials that travel from the brainstem to the spleen and other organs. This culminates in T cell release of acetylcholine, which interacts with α7 nicotinic acetylcholine receptors (α7nAChR) on immunocompetent cells to inhibit cytokine release in macrophages. This neural-immunomodulatory circuit, referred to as the “cholinergic anti-inflammatory pathway”, presents opportunities for developing novel therapeutic strategies to treat inflammatory diseases. It has been successfully implemented in models of inflammatory conditions like induced neuroinflammation, cerebral ischemia/reperfusion, rheumatoid arthritis, sepsis, and inflammatory bowel diseases or colitis. Harnessing its anti-inflammatory effects, VNS has been used in a mouse model of cerebral aneurysms and SAH. In this study, pre-treatment with VNS not only reduced the rupture rate of intracranial aneurysms, but also reduced the grade of hemorrhage if rupture occurred and improved survival and outcome after SAH. There has not been any work examining the effect of VNS on SAH in humans.

Historically, VNS was performed exclusively by surgical cervical neck dissection and placement of a cuff electrode directly around the nerve within the carotid sheath. Alternatively, VNS can be accomplished non-invasively by stimulating the auricular branch of the vagus nerve as it courses through the external ear, obviating the morbidity of a surgical procedure and allowing rapid deployment of the intervention in critically ill patients. The external ear is an ideal target for non-invasive stimulation of the vagus nerve, where the auricular branch travels in the concha of the ear.

Methods described herein are generally performed on a subject in need thereof. A subject in need of the therapeutic methods described herein can be a subject having, diagnosed with, suspected of having, or at risk for developing a spontaneous subarachnoid hemorrhage (SAH). A determination of the need for treatment will typically be assessed by a history, physical exam, or diagnostic tests consistent with the disease or condition at issue. Diagnosis of the various conditions treatable by the methods described herein is within the skill of the art. The subject can be an animal subject, including a mammal, such as horses, cows, dogs, cats, sheep, pigs, mice, rats, monkeys, hamsters, guinea pigs, and humans or chickens. For example, the subject can be a human subject.

Generally, a safe and effective amount of vagal nerve stimulation (VNS) is, for example, an amount that would cause the desired therapeutic effect in a subject while minimizing undesired side effects. In various embodiments, an effective amount of vagal nerve stimulation (VNS) described herein can substantially inhibit an inflammatory response, slow the progress of an inflammatory response, or limit the development of an inflammatory response associated with a spontaneous subarachnoid hemorrhage (SAH) in a subject.

The goal of the non-invasive ear stimulation (auricular branch of the vagus nerve) is to reduce the morbidity of subarachnoid hemorrhage. Reducing the morbidity can include, but is not limited to, reduction of hydrocephalus, reduction of vasospasm, reduction of infections, and/or reduction in ICU stay. Non-invasive ear stimulation can also be used to improve neurologic recovery from subarachnoid hemorrhage.

According to the methods described herein, the administration of VNS can be performed invasively or non-invasively. Non-limiting examples of suitable invasive methods for administering VNS include cervical neck dissection and placement of a cuff electrode directly on the vagus nerve. Non-limiting examples of suitable non-invasive methods for administering VNS include transcutaneous stimulation including, but not limited to, transcutaneous stimulation of the auricular branch of the vagus nerve using electrodes positioned on an ear of the subject.

The specific therapeutically effective dose level for any particular subject will depend upon a variety of factors including the disorder being treated and the severity of the disorder; the specific method of administration employed; the age, body weight, general health, sex, and diet of the subject; the time of administration; the route of administration; the duration of the treatment; drugs used in combination or coincidental with the specific method of administration employed; and like factors well known in the medical arts (see e.g., Koda-Kimble et al. (2004) Applied Therapeutics: The Clinical Use of Drugs, Lippincott Williams & Wilkins, ISBN 0781748453; Winter (2003) Basic Clinical Pharmacokinetics, 4th ed., Lippincott Williams & Wilkins, ISBN 0781741475; Shargel (2004) Applied Biopharmaceutics & Pharmacokinetics, McGraw-Hill/Appleton & Lange, ISBN 0071375503).

For example, it is well within the skill of the art to start doses of the composition at levels lower than those required to achieve the desired therapeutic effect and to gradually increase the dosage until the desired effect is achieved. If desired, the effective daily VNS dose may be divided into multiple doses for purposes of administration. Consequently, single dose VNS treatments may contain such amounts or submultiples thereof to make up the daily VNS dose. It will be understood, however, that the total daily usage of the VNS treatments of the present disclosure will be decided by an attending physician within the scope of sound medical judgment.

The systems and methods described herein are designed to benefit patients matching the states, diseases, disorders, and conditions described herein. Generally, treating a state, disease, disorder, or condition includes preventing, reversing, or delaying the appearance of clinical symptoms in a mammal that may be afflicted with or predisposed to the state, disease, disorder, or condition but does not yet experience or display clinical or subclinical symptoms thereof. Treating can also include inhibiting the state, disease, disorder, or condition, e.g., arresting or reducing the development of the disease or at least one clinical or subclinical symptom thereof. Furthermore, treating can include relieving the disease, e.g., causing regression of the state, disease, disorder, or condition or at least one of its clinical or subclinical symptoms. A benefit to a subject to be treated can be either statistically significant or at least perceptible to the subject or to a physician.

In some embodiments, administration of VNS can occur as a single event or over a time course of treatment. For example, VNS can be administered daily, weekly, bi-weekly, or monthly. For the treatment of acute conditions, the time course of treatment may be at least several days. Certain conditions could extend treatment from several days to several weeks. For example, treatment could extend over one week, two weeks, or three weeks. For more chronic conditions, treatment could extend from several weeks to several months or even a year or more.

Treatment in accord with the methods described herein can be performed prior to, concurrent with, or after conventional treatment modalities for subarachnoid hemorrhage (SAH).

VNS can be administered simultaneously or sequentially with another agent, such as an antibiotic, an anti-inflammatory, or another agent. For example, VNS can be administered simultaneously with another agent, such as an antibiotic or an anti-inflammatory. Simultaneous administration can occur through the administration of VNS along with separate compositions, each containing one or more of an antibiotic, an anti-inflammatory, or another agent. Simultaneous administration can occur through the administration of VNS along with one composition containing two or more of an antibiotic, an anti-inflammatory, or another agent. VNS can be administered sequentially with an antibiotic, an anti-inflammatory, or another agent. For example, VNS can be administered before or after administration of an antibiotic, an anti-inflammatory, or another agent.

To evaluate the effectiveness of vagal nerve stimulation (VNS) at reducing an inflammatory response associated with a spontaneous subarachnoid hemorrhage (SAH), the following experiments were conducted. Patients presenting with a spontaneous SAH were assigned to receive transcutaneous VNS using ear clip electrodes, or a sham treatment. Blood and CSF were collected from both groups and compared to assess the effect of VNS on various inflammatory biomarkers detected within the patient's blood and CSF samples.

The results of these experiments demonstrated an attenuation of the inflammatory response in those patients treated using VNS as compared to the patients receiving sham treatments

Definitions and methods described herein are provided to better define the present disclosure and to guide those of ordinary skill in the art in the practice of the present disclosure. Unless otherwise noted, terms are to be understood according to conventional usage by those of ordinary skill in the relevant art.

In some embodiments, numbers expressing quantities of ingredients, properties such as molecular weight, reaction conditions, and so forth, used to describe and claim certain embodiments of the present disclosure are to be understood as being modified in some instances by the term “about.” In some embodiments, the term “about” is used to indicate that a value includes the standard deviation of the mean for the device or method being employed to determine the value. In some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the present disclosure are approximations, the numerical values set forth in the specific examples are reported as precisely as practicable. The numerical values presented in some embodiments of the present disclosure may contain certain errors necessarily resulting from the standard deviation found in their respective testing measurements. The recitation of ranges of values herein is merely intended to serve as a shorthand method of referring individually to each separate value falling within the range. Unless otherwise indicated herein, each individual value is incorporated into the specification as if it were individually recited herein. The recitation of discrete values is understood to include ranges between each value.

In some embodiments, the terms “a” and “an” and “the” and similar references used in the context of describing a particular embodiment (especially in the context of certain of the following claims) can be construed to cover both the singular and the plural, unless specifically noted otherwise. In some embodiments, the term “or” as used herein, including the claims, is used to mean “and/or” unless explicitly indicated to refer to alternatives only or the alternatives are mutually exclusive.

Optional” or “optionally” means that the subsequently described event or circumstance may or may not occur, and that the description includes instances where the event occurs and instances where it does not.

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and can also cover other unlisted steps. Similarly, any composition or device that “comprises,” “has” or “includes” one or more features is not limited to possessing only those one or more features and can cover other unlisted features.

All methods described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the present disclosure and does not pose a limitation on the scope of the present disclosure otherwise claimed. No language in the specification should be construed as indicating any non-claimed element essential to the practice of the present disclosure.

Groupings of alternative elements or embodiments of the present disclosure disclosed herein are not to be construed as limitations. Each group member can be referred to and claimed individually or in any combination with other members of the group or other elements found herein. One or more members of a group can be included in, or deleted from, a group for reasons of convenience or patentability. When any such inclusion or deletion occurs, the specification is herein deemed to contain the group as modified thus fulfilling the written description of all Markush groups used in the appended claims.

Any publications, patents, patent applications, and other references cited in this application are incorporated herein by reference in their entirety for all purposes to the same extent as if each individual publication, patent, patent application, or other reference was specifically and individually indicated to be incorporated by reference in its entirety for all purposes. Citation of a reference herein shall not be construed as an admission that such is prior art to the present disclosure.

Having described the present disclosure in detail, it will be apparent that modifications, variations, and equivalent embodiments are possible without departing the scope of the present disclosure defined in the appended claims. Furthermore, it should be appreciated that all examples in the present disclosure are provided as non-limiting examples.

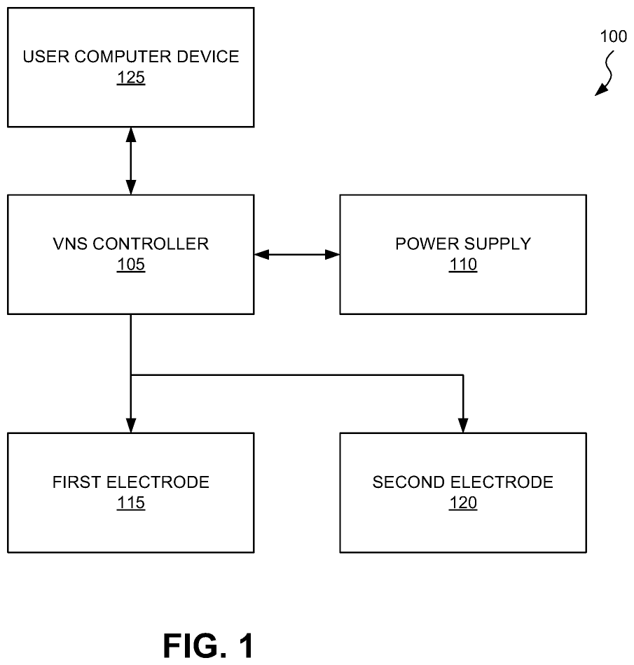

FIG. 1 illustrates a system 100 for providing vagal nerve stimulation to a patient in accordance with at least one embodiment.

The system 100 includes a VNS controller 105. The VNS controller 105 can be a computer device, such as a tablet, laptop, desktop, or other dedicated computer device including at least one processor in communication with at least one memory device. The VNS controller 105 can also include a user interface that that allows the VNS controller 105 to present information to a user and receive user inputs.

The VNS controller 105 is in communication with a power supply 110 configured to provide electrical stimulation. The VNS controller 105 can also be in communication with one or more electrodes, such as a first electrode 115 and a second electrode 120. The first electrode 115 and the second electrode 120 are configured to provide the electrical stimulation to the patient. In some embodiments, first electrode 115 and second electrode 120 are permanent, re-usable electrodes. In other embodiments first electrode 115 and second electrode 120 are disposable, single use electrodes. In still further embodiments, one or more of the first electrode 115 and the second electrode 120 are implanted in the patient to stimulate the vagus nerve. In additional embodiments, the first electrode 115 and the second electrode are temporarily attached to the patient's ear to stimulate the vagus nerve.

In at least one embodiment, the VNS controller 105 is configured to provide treatment to the vagus nerve by electrically stimulation for a period of twenty minutes. In at least one embodiment, the attributes of the electrical stimulation are 20 Hz, 250 μs, and 0.4 mA. In other embodiments, the current can range between 0.4 and 8 mA. The attributes of the electrical stimulation stay the same throughout the treatment. In at least one further embodiment, the electrical stimulation is performed twice a day. In at least one embodiment, the attributes of the electrical stimulation are selected to maximize vagus somatosensory evoked potentials while avoiding perception of pain.

In the exemplary embodiment, the VNS controller 105 controls the output of the power supply 110 to provide the electrical stimulation via the first electrode 115 and the second electrode 120.

In some further embodiments, VNS controller 105 is in communication with one or more user computer devices 125. The user computer device 125 may provide information to the VNS controller 105, such as one or more attributes of the patient that may alter the electrical stimulation applied to the patient. Furthermore, the user computer device 125 may provide timing information to the VNS controller 105, such as when to apply the electrical stimulation. Moreover, the user computer device 125 can receive information from the VNS controller 105, such as what were the attributes of the electrical stimulation that was applied to the patient.

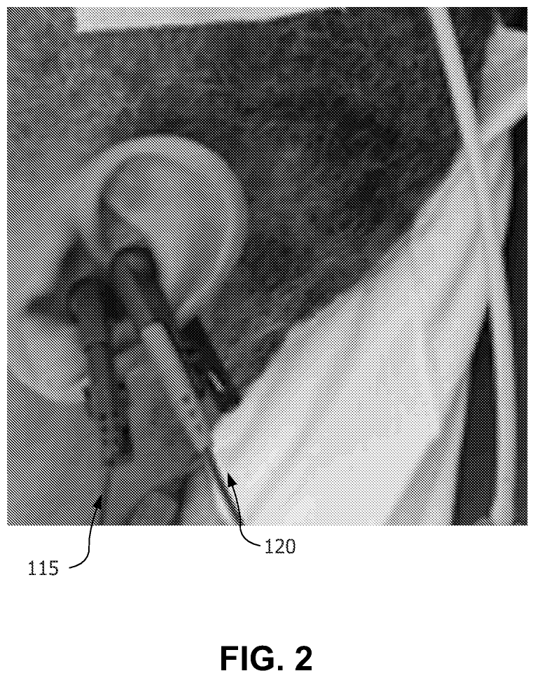

FIG. 2 illustrates placement of electrodes 115 and 120 (shown in FIG. 1) for non-invasive transcutaneous vagus nerve stimulation using the system 100 (shown in FIG. 1). In FIG. 2, the VNS controller 105 (shown in FIG. 1) is a part of a portable TENS (transcutaneous electrical nerve stimulation) unit. The TENS is connected to two the two electrodes 115 and 120.

In the exemplary embodiment, the first electrode 115 and the second electrode 120 are placed along the concha of the ear to stimulate the vagus nerve where the auricular branch travels in the pinna of the ear. In the exemplary embodiment, the first electrode 115 and the second electrode are attached to the patient's left ear.

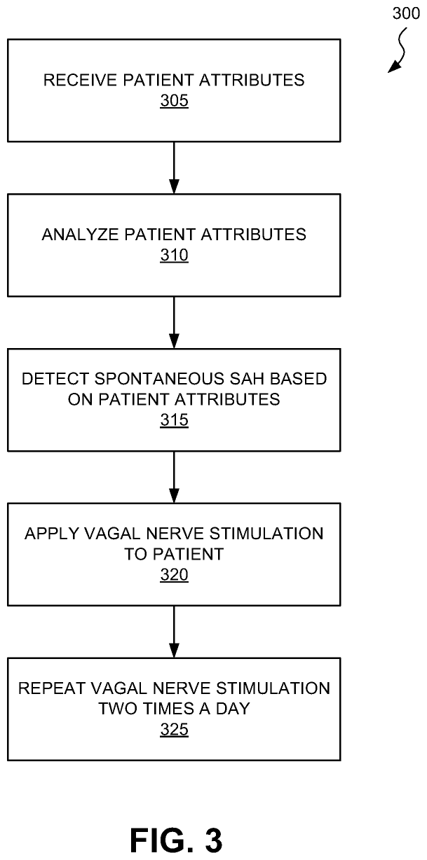

FIG. 3 illustrates a process 300 for providing vagal nerve stimulation using the system 100 (shown in FIG. 1). In the exemplary embodiment, portions of process 300 are performed by a user computer device 125 (shown in FIG. 1), which may be, but is not limited to, a tablet, a laptop, a desktop, and/or and other computer device including at least one processor in communication with at least one memory device. Additionally, portions of process 300 are performed by the VNS controller 105 (shown in FIG. 1).

In the exemplary embodiment, the user computer device 125 receives 305 patient attributes. The patient attributes could be received 305 when the patient checks in or by retrieving the patient history. The patient attributes can include but are not limited to, height, weight, gender, heart rate, blood pressure, medical history, reasons for admittance, bloodwork results, vital statistics, presence/location of an aneurysm on vascular imaging, Hunt and Hess grade of SAH, Fisher grade of SAH, and other attributes. The patient attributes can further include CT (Computed tomography) imaging of SAH with a cerebral aneurysm confirmed with a four-vessel cerebral angiogram. The patient attributes can be analyzed 310 to determine 315 if the patient is at risk for a spontaneous subarachnoid hemorrhage (SAH) based on the analyzed patient attributes.

If the patient is determined to be at risk for a SAH, the healthcare provider may apply 320 vagal nerve stimulation to the patient using the system 100 (shown in FIG. 1). The healthcare provider attaches two electrodes (first electrode 115 and second electrode 120 (shown in FIG. 1)) to the concha of the left ear of the patient. The VNS controller 105 then provides a current through the electrodes 115 and 120. In the exemplary embodiment, the current has the following attributes: 20 Hz, 250 μs, and 8 mA. The current can range from 0.4 mA to 8 mA. Furthermore, other attributes of the current can change depending on other factors, such as the attributes of the patient. The current is applied for a period of twenty minutes. Then the current is discontinued. In the exemplary embodiment, the electrical stimulation remains at the same attributes during the entire period of stimulation. In other embodiments, the electrical stimulation is started at a lower current and the VNS controller 105 increases the current over time.

In the exemplary embodiment, the healthcare provider and the VNS controller 105 repeat 325 the vagal nerve stimulation twice a day. After the electrical stimulation is complete, the patient's vital statistics can be monitored.

In the exemplary embodiment, the VNS controller 105 stimulates a patient's vagus nerve with an electrical signal to achieve a therapeutic effect for treating the inflammation, where the inflammation is related to a subarachnoid hemorrhage (SAH). The electrical signal includes a signal current of 0.4 mA, a pulse width of 250 μs, a signal frequency of 20 Hz, and a signal on-time of twenty minutes. The VNS controller 105 also re-stimulates the patient's vagus nerve a second time within 24 hours of a first stimulation. The electrical signal for the first stimulation and a second stimulation are the same and remain constant for the 20 minute duration of stimulation. In the exemplary embodiment, the stimulation is the transcutaneous stimulation of the vagus nerve, wherein the stimulation is provided via the first electrode 115 and the second electrode 120. The first electrode 115 and the second electrode 120 are attached to the concha of the patient's left ear. The stimulation is provided to the auricular branch of the vagus nerve where the vagus nerve travels in the pinna of the ear. In some further embodiments, the stimulation is paired with an antibiotic or an anti-inflammatory medication.

In addition to providing electrical stimulation to the patient, the healthcare provider also monitors multiple vital signs of the patient. In some embodiments, the patient's plasma and cerebrospinal fluid (CSF) are collected periodically, such as every three days, to quantify inflammatory markers. The rates of cerebral vasospasm and chronic hydrocephalus can be assessed. In addition, functional outcomes via modified Rankin Scale (mRS) scores can be collected.

In some embodiments, blood and Cerebrospinal fluid (CSF) samples are collected prior to the first electrical stimulation of the patient. The samples can be processed to provide the complete blood count with differential and CSF cell count with differential. For evaluation of the cytokines, the samples are centrifuged, aliquoted, and stored in a deep freezer until ready for processing.

Frozen supernatant plasma and CSF are slowly thawed and then analyzed in duplicate with multiplex kits (Thermofisher Scientific, Waltham, MA) for multiple pro-inflammatory cytokines: IL-1β, IL-2, IL-5, IL-6, IL-8, IL-12, IL-13, IL-17, TNF-α, GM-CSF, and IFN-γ; and anti-inflammatory cytokines: IL-4 and IL-10. The concentration of each antigen is calculated by plotting the expected concentration of the standards against the multiplex fluorescent immunoassay generated by each standard. A 4-parameter logistic regression is then used for the best-fit curve. Protein concentration is reported as pg/mL.

One goal is quantified continuous measures of the serum and CSF markers of inflammation (i.e., IL-6, TNF-α, etc.) collected at two time points, baseline (before treatment) and day 13 after treatment. The taVNS impact on SAH inflammatory markers can then be examined via a linear mixed model, where time (i.e., 0- and 13-days post-treatment), treatment (i.e., taVNS vs. Sham), and time-treatment interaction are the fixed effects, and the dependency of measurements clustered within each individual patient are accounted for.

In addition to analyze how the taVNS alters the development of the secondary SAH sequela of radiographic vasospasm, and its mediation by the inflammatory response, initial diagnostic imaging, patients will undergo a repeat computed tomography or catheter angiogram seven days after admission. Additionally, further vascular imaging will be performed if there is clinical concern per the intensive care or neurosurgical teams for clinical vasospasm or stroke. For both planned and indicated imaging sessions, each vascular imaging study is reviewed to describe the imaging as it relates to vasospasm as none, mild (<25% stenosis), moderate (25%-50% stenosis), or severe (>50% stenosis) narrowing of at least one major intracranial artery, as previously described. Additional clinical metrics related to vasospasm can also be used. Specifically, these can include the following: 1) blood pressure augmentation while in the intensive care unit, 2) number of vascular imaging sessions, 3) treatments performed during catheter angiogram (e.g., intraarterial vasodilators), 4) use of intrathecal vasodilators, and 5) CT imaging identified strokes and parenchymal volume of strokes.

The above analysis is to fully quantify the incidence, severity, and treatment response to radiographic vasospasm in SAH patients. The goal of the electrical stimulation treatment is to reduce radiographic vasospasm, as well as the need for vasospasm-related interventions like blood pressure augmentation, angioplasty, or intraarterial/intrathecal medications to negate spasm. The reduction of these findings can be associated with lowered CSF inflammatory makers. The taVNS can be correlated with lower blood pressure goals and reduced number of vasospasm interventions. These altered radiographic and clinical changes can also be correlated with a concomitant reduction in CSF inflammatory cytokines.

In a further embodiment, another goal includes defining how taVNS alters key clinical metrics associated with CSF malabsorption after SAH. taVNS can lead to a reduction in duration of EVD drainage and rate of ventricular shunting for chronic hydrocephalus. In this embodiment, specific details of a patient's clinical course as it relates to impaired CSF absorption and hydrocephalus is defined with primary outcome metrics including 1) need for surgical placement of permanent CSF diversion such as a ventriculoperitoneal or ventriculoatrial shunt prior to discharge from the hospital and 2) duration of external ventricular drainage.

The goal is to have SAH patients treated with taVNS to display a significant reduction in duration of EVD placement and lowered rates of chronic hydrocephalus requiring ventricular shunt. Further, these improvements correlate with reduced CSF inflammatory markers. This effect may be more pronounced in higher grade hemorrhage where the incidence of hydrocephalus is higher.

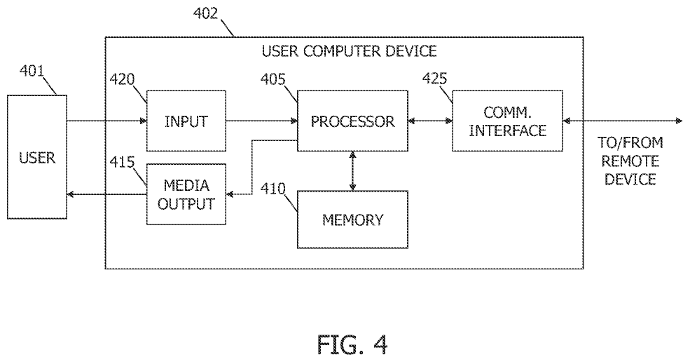

FIG. 4 illustrates an example configuration of a client system shown in FIG. 3, in accordance with one embodiment of the present disclosure. User computer device 402 is operated by a user 401. User computer device 402 may include, but is not limited to, VNS controller 105 and user computer device 125 (both shown in FIG. 1). User computer device 402 includes a processor 405 for executing instructions. In some embodiments, executable instructions are stored in a memory area 410. Processor 405 may include one or more processing units (e.g., in a multi-core configuration). Memory area 410 is any device allowing information such as executable instructions and/or transaction data to be stored and retrieved. Memory area 410 may include one or more computer-readable media.

User computer device 402 also includes at least one media output component 415 for presenting information to user 401. Media output component 415 is any component capable of conveying information to user 401. In some embodiments, media output component 415 includes an output adapter (not shown) such as a video adapter and/or an audio adapter. An output adapter is operatively coupled to processor 405 and operatively coupleable to an output device such as a display device (e.g., a cathode ray tube (CRT), liquid crystal display (LCD), light emitting diode (LED) display, or “electronic ink” display) or an audio output device (e.g., a speaker or headphones). In some embodiments, media output component 415 is configured to present a graphical user interface (e.g., a web browser and/or a client application) to user 401. A graphical user interface may include, for example, patient attributes or the attributes of the electrical stimulation. In some embodiments, user computer device 402 includes an input device 420 for receiving input from user 401. User 401 may use input device 420 to, without limitation, select to apply the electrical stimulation to the patient. Input device 420 may include, for example, a keyboard, a pointing device, a mouse, a stylus, a touch sensitive panel (e.g., a touch pad or a touch screen), a gyroscope, an accelerometer, a position detector, a biometric input device, and/or an audio input device. A single component such as a touch screen may function as both an output device of media output component 415 and input device 420.

User computer device 402 may also include a communication interface 425, communicatively coupled to a remote device such as a VNS controller 105 or a user computer device 125. Communication interface 425 may include, for example, a wired or wireless network adapter and/or a wireless data transceiver for use with a mobile telecommunications network.

Stored in memory area 410 are, for example, computer-readable instructions for providing a user interface to user 401 via media output component 415 and, optionally, receiving and processing input from input device 420. The user interface may include, among other possibilities, a web browser and/or a client application. Web browsers enable users, such as user 401, to display and interact with media and other information typically embedded on a web page or a website provided by a server. A client application allows user 401 to interact with, for example, VNS controller 105. For example, instructions may be stored by a cloud service and the output of the execution of the instructions sent to the media output component 415.

A computer program of one embodiment is embodied on a computer-readable medium. In an example, the system is executed on a single computer system, without requiring a connection to a server computer. In a further example embodiment, the system is being run in a Windows® environment (Windows is a registered trademark of Microsoft Corporation, Redmond, Washington). In yet another embodiment, the system is run on a mainframe environment and a UNIX® server environment (UNIX is a registered trademark of X/Open Company Limited located in Reading, Berkshire, United Kingdom). In a further embodiment, the system is run on an iOS® environment (iOS is a registered trademark of Cisco Systems, Inc. located in San Jose, CA). In yet a further embodiment, the system is run on a Mac OS® environment (Mac OS is a registered trademark of Apple Inc. located in Cupertino, CA). In still yet a further embodiment, the system is run on Android® OS (Android is a registered trademark of Google, Inc. of Mountain View, CA). In another embodiment, the system is run on Linux® OS (Linux is a registered trademark of Linus Torvalds of Boston, MA). The application is flexible and designed to run in various different environments without compromising any major functionality. In some embodiments, the system includes multiple components distributed among a plurality of computing devices. One or more components are in the form of computer-executable instructions embodied in a computer-readable medium. The systems and processes are not limited to the specific embodiments described herein. In addition, components of each system and each process can be practiced independently and separately from other components and processes described herein. Each component and process can also be used in combination with other assembly packages and processes.

As used herein, the terms “processor” and “computer” and related terms, e.g., “processing device”, “computing device”, and “controller” are not limited to just those integrated circuits referred to in the art as a computer, but broadly refers to a microcontroller, a microcomputer, a programmable logic controller (PLC), an application specific integrated circuit (ASIC), and other programmable circuits, and these terms are used interchangeably herein. In the embodiments described herein, memory may include, but is not limited to, a computer-readable medium, such as a random-access memory (RAM), and a computer-readable non-volatile medium, such as flash memory. Alternatively, a floppy disk, a compact disc-read only memory (CD-ROM), a magneto-optical disk (MOD), and/or a digital versatile disc (DVD) may also be used. Also, in the embodiments described herein, additional input channels may be, but are not limited to, computer peripherals associated with an operator interface such as a mouse and a keyboard. Alternatively, other computer peripherals may also be used that may include, for example, but not be limited to, a scanner. Furthermore, in the exemplary embodiment, additional output channels may include, but not be limited to, an operator interface monitor.

Further, as used herein, the terms “software” and “firmware” are interchangeable and include any computer program storage in memory for execution by personal computers, workstations, clients, servers, and respective processing elements thereof.

As used herein, the term “non-transitory computer-readable media” is intended to be representative of any tangible computer-based device implemented in any method or technology for short-term and long-term storage of information, such as, computer-readable instructions, data structures, program modules and sub-modules, or other data in any device. Therefore, the methods described herein may be encoded as executable instructions embodied in a tangible, non-transitory, computer readable medium, including, without limitation, a storage device, and a memory device. Such instructions, when executed by a processor, cause the processor to perform at least a portion of the methods described herein. Moreover, as used herein, the term “non-transitory computer-readable media” includes all tangible, computer-readable media, including, without limitation, non-transitory computer storage devices, including, without limitation, volatile and nonvolatile media, and removable and non-removable media such as a firmware, physical and virtual storage, CD-ROMs, DVDs, and any other digital source such as a network or the Internet, as well as yet to be developed digital means, with the sole exception being a transitory, propagating signal.

Furthermore, as used herein, the term “real-time” refers to at least one of the time of occurrence of the associated events, the time of measurement and collection of predetermined data, the time for a computing device (e.g., a processor) to process the data, and the time of a system response to the events and the environment. In the embodiments described herein, these activities and events may be considered to occur substantially instantaneously.

The computer-implemented methods and processes described herein may include additional, fewer, or alternate actions, including those discussed elsewhere herein. The present systems and methods may be implemented using one or more local or remote processors, transceivers, and/or sensors (such as processors, transceivers, and/or sensors mounted on vehicles, stations, nodes, or mobile devices, or associated with smart infrastructures and/or remote servers), and/or through implementation of computer-executable instructions stored on non-transitory computer-readable media or medium. Unless described herein to the contrary, the various steps of the several processes may be performed in a different order, or simultaneously in some instances.

Additionally, the computer systems discussed herein may include additional, fewer, or alternative elements and respective functionalities, including those discussed elsewhere herein, which themselves may include or be implemented according to computer-executable instructions stored on non-transitory computer-readable media or medium.

In the exemplary embodiment, a processing element may be instructed to execute one or more of the processes and subprocesses described above by providing the processing element with computer-executable instructions to perform such steps/sub-steps, and store collected data (e.g., trust stores, authentication information, etc.) in a memory or storage associated therewith. This stored information may be used by the respective processing elements to make the determinations necessary to perform other relevant processing steps, as described above.

The aspects described herein may be implemented as part of one or more computer components, such as a client device, system, and/or components thereof, for example. Furthermore, one or more of the aspects described herein may be implemented as part of a computer network architecture and/or a cognitive computing architecture that facilitates communications between various other devices and/or components. Thus, the aspects described herein address and solve issues of a technical nature that are necessarily rooted in computer technology.

Although specific features of various embodiments may be shown in some drawings and not in others, this is for convenience only. In accordance with the principles of the systems and methods described herein, any feature of a drawing may be referenced or claimed in combination with any feature of any other drawing.

Some embodiments involve the use of one or more electronic or computing devices. Such devices typically include a processor, processing device, or controller, such as a general purpose central processing unit (CPU), a graphics processing unit (GPU), a microcontroller, a reduced instruction set computer (RISC) processor, an application specific integrated circuit (ASIC), a programmable logic circuit (PLC), a programmable logic unit (PLU), a field programmable gate array (FPGA), a digital signal processing (DSP) device, and/or any other circuit or processing device capable of executing the functions described herein. The methods described herein may be encoded as executable instructions embodied in a computer readable medium, including, without limitation, a storage device and/or a memory device. Such instructions, when executed by a processing device, cause the processing device to perform at least a portion of the methods described herein. The above examples are exemplary only, and thus are not intended to limit in any way the definition and/or meaning of the term processor and processing device.

The computer-implemented methods discussed herein may include additional, less, or alternate actions, including those discussed elsewhere herein. The methods may be implemented via one or more local or remote processors, transceivers, servers, and/or sensors, and/or via computer-executable instructions stored on non-transitory computer-readable media or medium.

Additionally, the computer systems discussed herein may include additional, less, or alternate functionality, including that discussed elsewhere herein. The computer systems discussed herein may include or be implemented via computer-executable instructions stored on non-transitory computer-readable media or medium.

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims.

What is claimed is:

1. A method of treating an inflammation in a patient, the method comprising:
stimulating, in a patient suffering from inflammation, a patient's vagus nerve with an electrical signal to achieve a therapeutic effect for treating the inflammation, wherein a signal current is 0.4 mA, a pulse width is 250 μs, a signal frequency is 20 Hz, and a signal on-time is twenty minutes; and
re-stimulating the patient's vagus nerve a second time within 24 hours of a first stimulation, wherein the electrical signal for the first stimulation and a second stimulation are the same.

2. The method of claim 1, wherein the stimulation is transcutaneous stimulation of the vagus nerve.

3. The method of claim 2, wherein the stimulation is provided via a first electrode and a second electrode, wherein the first electrode and the second electrode are attached to the concha of the patient's ear.

4. The method of claim 3, wherein the first electrode and the second electrode are attached to the patient's left ear.

5. The method of claim 3, wherein the stimulation is provided to the auricular branch of the vagus nerve where the vagus nerve travels in the pinna of the ear.

6. The method of claim 1, wherein the stimulation is paired with an antibiotic or an anti-inflammatory medication.

7. The method of claim 1, wherein the stimulation is provided via an implanted cuff electrode placed directly on the vagus nerve.

8. The method of claim 1, wherein the inflammation is related to a subarachnoid hemorrhage (SAH).

9. The method of claim 1 further comprising:
receiving a plurality of patient attributes associated with the patient;
analyzing the plurality of patient attributes; and
determining that the patient is at risk for a spontaneous subarachnoid hemorrhage (SAH) based on the analyzed patient attributes.

10. A system for treating an inflammation in a patient, the system comprising an electrical stimulation device including one or more electrodes, wherein the electrical stimulation device is configured to provide an electrical current to a patient's vagus nerve with an electrical signal to achieve a therapeutic effect for treating inflammation, wherein a signal current is 0.4 mA, a pulse width is 250 μs, a signal frequency is 20 Hz, and a signal on-time is twenty minutes.

11. The system of claim 10, wherein the patient is suffering from inflammation.

12. The system of claim 10, wherein the inflammation is related to a subarachnoid hemorrhage (SAH).

13. The system of claim 10, wherein the electrical stimulation device is further configured to re-stimulate the patient's vagus nerve a second time within 24 hours of a first stimulation, wherein the electrical signal for the first stimulation and a second stimulation are the same.

14. The system of claim 10, wherein the stimulation is transcutaneous stimulation of the vagus nerve.

15. The system of claim 14, wherein the stimulation is provided via a first electrode and a second electrode, wherein the first electrode and the second electrode.

16. The system of claim 15, wherein the first electrode and the second electrode are attached to the patient's left ear.

17. The system of claim 15, wherein the stimulation is provided to the auricular branch of the vagus nerve where the vagus nerve travels in the pinna of the ear.

18. The system of claim 10, wherein the stimulation is paired with an antibiotic or an anti-inflammatory medication.

19. The system of claim 10, wherein the stimulation is provided via an implanted cuff electrode placed directly on the vagus nerve.

20. An electrical stimulation device including one or more electrodes, wherein the electrical stimulation device is configured to provide an electrical current to a patient's vagus nerve with an electrical signal to achieve a therapeutic effect for treating inflammation, wherein a signal current is 0.4 mA, a pulse width is 250 μs, a signal frequency is 20 Hz, and a signal on-time is twenty minutes.
